(19) United States
(12) Patent Application Publication
(10) Pub. No.: US 2014/0343463 A1
(43) Pub. Date: Nov. 20, 2014

(54) ULTRASOUND NEUROMODULATION TREATMENT OF CLINICAL CONDITIONS

(71) Applicant: David J. Mishelevich, Playa del Rey, CA (US)

(72) Inventor: David J. Mishelevich, Playa del Rey, CA (US)

(21) Appl. No.: 13/898,401

(22) Filed: May 20, 2013

(51) Int. Cl. A61N 7/00

(52) U.S. Cl. CPC A61N 7/00
USPC 601/2

(57) ABSTRACT

Disclosed are methods and systems for non-invasive ultrasound neuromodulation of neural targets for the treatment of clinical conditions. These include neuromodulation of the occipital nerves to treat migraine and cluster headaches in their multiple variations as well other pain and tension conditions, the Sphenopalatine Ganglion and associated neural structures vidian nerve and/or sphenopalatine nerve to treat migraine and cluster headaches as well as other indications such as neurologic and psychiatric conditions, and the Reticular Activating System for a variety of clinical purposes such as reversibly putting a patient to sleep or waking them up (for example, for the purpose of anesthesia) or reversibly putting a patient into a coma (for example for the purpose of protecting or rehabilitating the brain of the patient after a stroke or head injury). Other clinical applications include neuromodulation of the Motor Cortex and other areas of the brain impacted by stroke for mitigating the effects of stroke and/or in stroke rehabilitation, pain-related targets to treat acute or chronic pain, tinnitus-related targets to treat that condition, and targets for the treatment of Post-Traumatic Stress Disorder. Use of ultrasound neuromodulation in sessions can enhance the effects.

### CROSS REFERENCE TO RELATED APPLICATIONS

This patent application claims priority as a continuation-in-part of patent applications U.S. patent application Ser. No. 13/021,785, titled “ULTRASOUND NEUROMODULATION OF THE OCCIPUT,” filed on Feb. 7, 2011; U.S. patent application Ser. No. 13/020,016, titled “ULTRASOUND NEUROMODULATION OF THE SPHENOPALATINE GANGLION,” filed on Feb. 3, 2011; U.S. patent application Ser. No. 13/031,192, titled “ULTRASOUND NEUROMODULATION OF THE RETICULAR ACTIVATING SYSTEM,” filed on Feb. 19, 2011; U.S. patent application Ser. No. 13/405,337, titled “ULTRASOUND NEUROMODULATION FOR STROKE MITIGATION AND REHABILITATION,” filed on Feb. 26, 2012; U.S. patent application Ser. No. 13/411,641, titled “ULTRASOUND NEUROMODULATION TREATMENT OF PAIN,” filed on Mar. 5, 2012; U.S. patent application Ser. No. 13/413,659, titled “ULTRASOUND NEUROMODULATION TREATMENT OF TINNITUS,” filed on Mar. 7, 2012; and U.S. patent application Ser. No. 13/430,729, titled “ULTRASOUND NEUROMODULATION TREATMENT OF POST TRAUMATIC STRESS DISORDER,” filed on Mar. 27, 2012. This application also claims priority to U.S. Patent Application No. 61/666,825, titled “ULTRASOUND NEUROMODULATION DELIVERED IN SESSIONS”, filed on Jun. 30, 2012.

This patent application may be related to one or more of the following patents and pending patent applications (US and PCT applications), each of which is herein incorporated by reference in its entirety: U.S. patent application Ser. No. 12/940,052, titled “NEUROMODULATION OF DEEP-BRAIN TARGETS USING FOCUSED ULTRASOUND,” filed on Nov. 5, 2010; U.S. patent application Ser. No. 12/958,411, titled “MULTI-MODALITY NEUROMODULATION OF BRAIN TARGETS,” filed on Dec. 2, 2010; U.S. patent application Ser. No. 13/007,626, titled “PATIENT FEEDBACK FOR CONTROL OF ULTRASOUND DEEP-BRAIN NEUROMODULATION,” filed on Jan. 15, 2011; U.S. patent application Ser. No. 13/200,903, titled “SHAPED AND STEERED ULTRASOUND FOR DEEP-BRAIN NEUROMODULATION,” filed on Jan. 15, 2011; U.S. patent application Ser. No. 13/098,473, titled “ULTRASOUND MACRO-PULSE AND MICRO-PULSE SHAPES FOR NEUROMODULATION,” filed on May 1, 2011; U.S. patent application Ser. No. 13/252,054, titled “ULTRASOUND-INTERSECTING BEAMS FOR DEEP-BRAIN NEUROMODULATION,” filed on Oct. 3, 2011; and U.S. patent application Ser. No. 13/360,600, titled “PATTERNED CONTROL OF ULTRASOUND FOR NEUROMODULATION,” filed on Jan. 27, 2012.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation of neural structures for the purpose of treatment of clinical conditions.

### BACKGROUND OF THE INVENTION

It has been demonstrated that focused ultrasound directed at neural structures can stimulate those structures. If neural activity is increased or excited, the neural structure is said to be up regulated; if neural activated is decreased or inhibited, the neural structure is said to be down regulated. One or a plurality of neural elements can be neuromodulated.

Potential application of ultrasonic therapy of deep-brain structures has been covered previously (Gavrilov L R, Tsirulnikov E M, and IA Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). It was noted that monophasic ultrasound pulses are more effective than biphasic ones.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Patent applications have been filed addressing neuromodulation of deep-brain targets (Bystritsky, “Methods for modifying electrical currents in neuronal circuits,” U.S. Pat. No. 7,283,861, Oct. 16, 2007 and Deisseroth, K. and M. B. Schneider, “Device and method for non-invasive neuromodulation,” U.S. patent application Ser. No. 12/263,026 published as US 2009/0112133 A1, Apr. 30, 2009).

While sessions have been used in the application of Transcranial Magnetic Stimulation, this has not been done with ultrasound neuromodulation.

Transcranial Magnetic Stimulation (TMS) has been successfully used in occipital nerve stimulation for migraine headache and other headaches. For example in Mohammed et al. (Mohammad, Y. M., Kothari, R., Hughes, G., Nkrumah, M., Fischell, S., Fischell, R. F., Schweiger, J., and P. Ruppel, “Transcranial Magnetic Stimulation (TMS) relieves migraine headache,” Abstract, American Headache Society Meeting 2006), two TMS pulses were delivered, 30 seconds apart. The treatment was well tolerated and there was a tendency to reduce pain at two hours as well as nausea and cognitive function in the double blind, placebo controlled study. In a single pulse TMS study Lipton et al. (Lipton, R. B., Dodick, D. W., Goadsby, P. J., Saper, J. R., Silberstein, S. D., Aurora S. K., Mohammad, Y. M.; Ruppel, P. L., and R. E. Fischell, “Transcranial Magnetic Stimulation (TMS) Using a Portable Device is Effective for the Acute Treatment of Migraine with Aura: Results of a Double Blind, Sham Controlled, Randomized Study,” Abstract, American Headache Society Meeting June 2008) in which there was also relief at two hours. In another study Clarke et al. (Clarke, B. A., Upton, A. R. M., Kamath, M. V., Al-Harbi, T., and C. M. J. Castellanos. “Transcranial magnetic stimulation for migraine: clinical effects,” Headache Pain, 7:341-346, 2006) involving two-pulse stimulation of the autonomic nervous system to treat migraine headache, if patient had aura, improvement was typically immediate majority of patients got relief with no adverse side effects.

Deep-brain stimulation (DBS) of the occipital nerves has also been used to treat headache and other maladies. For example, Burns et al. studied cluster headaches (Burns, B., Watkins, L., and P. Goadsby, “Treatment of medically intractable cluster headache by occipital nerve stimulation: long-term follow-up of eight patients,” The Lancet, Volume 369, Issue 9567, Pages 1099-1106, 31 Mar. 2007). Seven of the patients were bilaterally stimulated and one unilaterally stimulated and six of the eight patients reported meaning responses with improvement in both frequency and severity of attacks.

Autonomic stimulation to positively impact intracranial structures such as the Vagal Nerve Stimulation (VNS) is used successfully in clinical practice (e.g., George, M., Sackheim, A J, Rush, et al., “Vagus Nerve Stimulation: A New Tool for Brain Research and Therapy,” Biological Psychiatry, 47, 287-295, 2000).

Electrical stimulation, including autonomic nervous system stimulation, has been associated with treatment of headaches and associated symptoms such as nausea and vomiting. A variety of non-invasive treatments have been used for headache treatment such as medication, diet, trigger avoidance, acupuncture, anesthetic agents, biofeedback, and physical therapy. Invasive treatments have been used as well such as ganglion resection, ganglion block, radiosurgery, and cryotherapy. Electrical stimulation has been applied by implanted electrodes or implanted stimulator. A stimulator can be set to deliver a predetermined pattern of stimulation, or the patient may control the amplitude, pulse width, and frequency using a remote-control device.

Such stimulation has also been associated with the treatment of a number of other conditions including neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders.

Many of the sensory and motor nerves of the neck are contained in C2 and C3, including the Greater Occipital Nerve (GON). NeurologyReviews.com (Vol. 16, Num. 10, October 2008) reviewed considerations of stimulation of the occipital nerve in treatments of headaches such as migraine, cluster, and hemicrania continua. Blocks of the occipital nerve have had success in treatment of headache in its various forms. An important aspect is that positive effect of the treatment outlasts the impact of the neural block. This indicates that there is some longer-term neuromodulation. Such blocks, while effective in a majority of cases, are not always predictive of whether longer-term occipital nerve electrical stimulation will be successful. In some cases, there is a delayed effect (which may be two to six months and may involve the patient's symptoms getting worse before they get better) so a short-term trial stimulation does not mean longer-term stimulation will not be successful. The length of time to achieve therapeutic effect means that the mechanism of impact involves neural plasticity. Also that anterior-pain symptoms decrease as well as posterior-pain symptoms indicates that a central mechanism is involved. In addition, for hemicrania continua, pain remediation may be separate from autonomic symptoms such as rhinorrhea and tearing excess that can remain after headache symptoms decrease. Meningeal and Greater Occipital Nerve inputs come together, not peripherally but centrally at the second-order neuron in the spinal cord (Bartsch, T. and P. J. Goadsby, “Stimulation of the greater occipital nerve induces increased central excitability of the dural afferent input,” Brain, 125:1496-1509, 2002.) indicating involvement of the caudal trigeminal nucleus and the upper cervical segments and suggesting a mechanism for referred pain.

A suggested mechanism for the etiology of headache is sensitization of the brainstem because of the sensory input from the occipital nerve causing altered neural processing (Muehlberger, T., Brittner, W., Buschmann, A., and T. Nidal Toman, “Lasting Outcome of the Surgical Treatment of Migraine Headaches—a Four Year Follow-up,” Abstract #14728, Meeting of the American Society of Plastic Surgery, Nov. 3, 2008).

For the treatment of migraine and cluster headaches and other conditions, it would be of benefit to apply a non-invasive treatment modality.

While Transcranial Magnetic Stimulation (TMS) is an effective means of non-invasive neuromodulation when used intracranially, current systems have delivered footprints that are too large for neural structures like the Sphenopalatine Ganglion. Ultrasound can be focused to 0.5 to 2 mm while TMS can be focused to 1 cm at best. Also, if TMS were used to stimulate the Sphenopalatine Ganglion there would be intolerable side effects such local muscle stimulation, and, in some cases stimulation of other nerves.

Autonomic stimulation to positively impact intracranial structures such as the Vagal Nerve Stimulation (VNS) is used successfully in clinical practice (e.g., George, M., Sackheim, A J, Rush, et al., “Vagus Nerve Stimulation: A New Tool for Brain Research and Therapy,” Biological Psychiatry, 47, 287-295, 2000).

Sphenopalatine Ganglion and other autonomic nervous system stimulation has been associated with treatment of headaches and associated symptoms such as nausea and vomiting. A variety of non-invasive treatments have been used for headache treatment such as medication, diet, avoidance of triggers, acupuncture, anesthetic agents, biofeedback, and physical therapy. Invasive treatments have been used as well such as ganglion resection, ganglion block, radiosurgery, and cryotherapy. In addition, electrical stimulation has been applied by implanted electrodes or implanted stimulator.

Such stimulation has also been associated with the treatment of a number of other conditions including neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders.

In addition, stimulation of the Sphenopalatine Ganglion has been described for modification the properties of the Blood Brain Barrier (BBB) and cerebral blood flow (Shalev, A. and Y. Gross, “Method and apparatus for stimulating the sphenopalatine ganglion to modify properties of the BBB and cerbral blood flow,” U.S. Pat. No. 7,190,998, Issued Mar. 13, 2007).

The sphenopalatine ganglion is a parasympathetic ganglion the largest of the parasympathetic ganglia associated with the branches of the trigeminal nerve. Stimulation of the Sphenopalatine Ganglion (SPG) for a number of maladies has been addressed previously. Examples are Pless (B. D. Pless, “Method and Device for the Treatment of Headache,” U.S. Patent Application Pub. No. 2009/0276005), Yun and Lee (Yun, A. J., and P. Y-b Lee, “Treatment of conditions through modulation of the autonomic nervous system,” U.S. Pat. No. 7,363,076), and Ansarinia (Ansarinia, M. M., “Stimulation Method for the Sphenopalatine Ganglia, Sphenopalatine Nerve, or Vidian Nerve for Treatment of Medical Conditions,” U.S. Pat. No. 6,526,318, Feb. 25, 2003). It would desirable to have neuromodulation of the SPG and related structures without using invasive means such as implanted electrodes.

While ultrasound can be focused down to a diameter on the order of one to a few millimeters (depending on the frequency), whether such a tight focus is required depends on the conformation of the neural target. For example, some targets, like the Reticular Activating System, are elongated and will be more effectively served with an elongated ultrasound field at the target.

The primary auditory cortex is essentially in the same region as Brodmann areas 41 and 42. It is located in the posterior half of the superior temporal gyms and also dives into the lateral sulcus as the transverse temporal gyri. Neuromodulation of the Primary Auditory Cortex (PAC) using repetitive Transcranial Magnetic Stimulation (rTMS) for a week at 1 Hz. demonstrated the elimination or reduction of tinnitus in over 50% of the patients (Rossi S, De Capua A, Ulivelli M, et al. Effects of repetitive transcranial magnetic stimulation on chronic tinnitus: a randomized, crossover, double blind, placebo controlled study. J Neurol Neurosurg Psychiatry. 2007; 78(8):857-863.).

Kleinjung et al. (Kleinjung T, Steffens T, Londero A, Langguth B, “Transcranial magnetic stimulation (TMS) for treatment of chronic tinnitus: clinical effects,” Prog Brain Res. 2007; 166:359-67) located the target that they used for TMS stimulation by looking at areas of increased metabolic activity demonstrated in chronic tinnitus patients by PET imaging using 18F deoxyglucose (FDG) and fusing the images with structural MRI scans to obtain anatomic correlations. The same targets would be used for ultrasound neuromodulation. The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

Multiple targets can be neuromodulated singly or in groups to treat PTSD. To accomplish the treatment, in some cases the neural targets will be up regulated and in some cases down regulated, depending on the given neural target. Targets have been identified by such methods as PET imaging, fMRI imaging, and clinical response to Transcranial Magnetic Stimulation (TMS).

For treatment of PTSD, primary neural targets are the Amygdala, Hippocampus, Anterior Cingulate Cortex, Orbito-Frontal Cortex, and the Insula. An additional target can be the Ventro-Medial Pre-Frontal Cortex and others may be discovered as well. One consideration is that PTSD may involve dysfunction of the Hypothalamic, pituitary-adrenal axis involving the Hippocampus, Amygdala, and Pre-Frontal Cortex (PFC) as in Ruiz et al. (Ruiz J E, Barbosa Neto J, Schoedl A F, and MF Mello M F, “Psychoneuroendocrinology of posttraumatic stress disorder,” Rev Bras Psiquiatr. 2007 May; 29 Suppl. 1:S7-12.).

In the application of the therapeutic ultrasound, the hyperactive Amygdala would be down regulated, the Anterior Cingulate Cortex (ACC) up regulated, the Orbito-Frontal Cortex (OFC) up regulated, the Hippocampus up regulated, and the Insula down regulated. If the Ventro-Medical Pre-Frontal Cortex were targeted it would be up regulated.

The Amygdala, Anterior Cingulate Cortex, Orbito-Frontal Cortex, and the Hippocampus targets were identified in Jatzko et al. (Jatzko A, Schmitt A, Kordon A, and DF Braus D F, “Neuroimaging findings in posttraumatic stress disorder: review of the literature,” Fortschr Neurol Psychiatr. 2005 July; 73(7):377-91.). Involvement of the Amygdala, and Anterior Cingulate Cortex plus addition of the Insula was identified in Liberzon et al. (Liberzon I, Britton J C, and K L Phan K L, “Neural correlates of traumatic recall in posttraumatic stress disorder,” Stress. 2003 September; 6(3):151-6.).

### SUMMARY OF THE INVENTION

It is the purpose of this invention to provide methods and systems and methods for ultrasound neuromodulation of the occipital nerves, Sphenopalatine Ganglion, Reticular Activating System, selected portions of the brain to mitigate against the impacts of stroke and foster stroke rehabilitation, treatment of acute and chronic pain, treatment of tinnitus, and treatment of Post-Traumatic Stress Disorder.

Effectiveness of the treatment can be enhanced by the application of the ultrasound neuromodulation in sessions. An example of sessions is neuromodulation for 30 minutes per day for five days a week over a two-week period. Most applications will not be amenable to continuous neuromodulation. Application of patterned ultrasound neuromodulation can enhance the effects.

### BRIEF DESCRIPTION OF THE DRAWINGS

FIG. 1 shows a diagram of exemplar session types for both initial treatment and maintenance sessions.

FIG. 2 shows an ultrasound transducer against the occiput of the patient targeting the occipital nerves using an embodiment using either a unilateral ultrasound transducer or a pair of ultrasound transducers for bilateral stimulation.

FIG. 3 shows a diagram of the occipital nerves relative to the occiput.

FIG. 4 shows a block diagram of the control circuit.

FIG. 5. shows an ultrasound transducer against the face of the patient targeting the Sphenopalatine Ganglion and related neural structures.

FIG. 6 shows a diagram of the Sphenopalatine Ganglion and related neural structures.

FIG. 7 illustrates the anatomic relationships of the Sphenopalatine Ganglion and related neural structures with the bony structures of the face.

FIG. 8 shows sagittal view of brain highlighting the Reticular Activating System including ultrasound transducer positioning.

FIG. 9 illustrates two alternative ultrasound transducer positions for targeting the Reticular Activating System.

FIG. 10 shows side and top views of pattern generated by the ultrasound transducer.

FIG. 11 with respect to the treatment of stroke shows a lateral view of the brain with the location of the motor cortex indicated.

FIG. 12 illustrates two different views of an ultrasound transducer that produces an elongated beam to neuromodulate elongated targets.

FIG. 13 demonstrates the positioning of an ultrasound transducer relative to the motor cortex.

FIG. 14 with respect to the treatment of pain shows ultrasonic-transducer targeting of the Rostral Anterior Cingulate Cortex (ACC) and the Dorsal Anterior Cingulate Gyms (DACG).

FIG. 15 with respect to the treatment of tinnitus shows ultrasonic-transducer targeting of the Primary Auditory Cortex (PAC) within the Temporal Lobe.

FIG. 16 with respect to the treatment of Post-Traumatic Stress Disorder (PTSD), shows ultrasonic-transducer targeting of the Orbito-Frontal Cortex (OFC), the Anterior Cingulate Cortex (ACC), the Insula, the Amygdala, and the Hippocampus.

### DETAILED DESCRIPTION OF THE INVENTION

It is the purpose of this invention to provide methods and systems and methods for ultrasound neuromodulation of the occipital nerves, Sphenopalatine Ganglion, Reticular Activating System, selected portions of the brain to mitigate against the impacts of stroke and foster stroke rehabilitation, treatment of acute and chronic pain, treatment of tinnitus, and treatment of Post-Traumatic Stress Disorder. In addition, effectiveness of the treatment can be enhanced by the application of the ultrasound neuromodulation in sessions and also the use of stimulation patterns. Most applications will not be amenable to continuous neuromodulation.

With respect to delivery of ultrasound neuromodulation in sessions, examples of session types include periodic sessions over extended time typically means a single session of length on the order of 15 to 60 minutes repeated daily or five days per week over one to six weeks. Other lengths of session or number of weeks of neuromodulation are applicable, such as session lengths up to 2.5 hours and number of weeks ranging from one to eight. Period sessions over compressed time typically means a single session of length on the order of 30 to 60 minutes repeated during awake hours with inter-session times of 15 minutes to 60 minutes over one to three days. Other inter-session times such as 15 minutes to three hours and days of compressed therapy such as one to five days are applicable. Maintenance consists of periodic sessions at fixed intervals or on as-needed maintenance tune-ups. Maintenance categories are maintenance post-completion of original treatment at fixed intervals and maintenance post-completion of original treatment with as-needed maintenance tune-ups. An example of the former are with one or more 50-minutes sessions during week 2 of months four and eight, and of the latter is one or more 50-minute sessions during week 7 because a tune up is needed at that time as indicated by return of symptoms. Use of sessions is important for the retraining of neural pathways for change of function, maintenance of function, or restoration of function. Retraining over time, with its ongoing reinforcement, can allow more effectively achievement of desired impacts. Another consideration is the desirability for practical reasons to limit tying up the time of the patient depending on the individual situation. Such neuromodulation systems can produce applicable acute or long-term effects. The latter occur through Long-Term Depression (LTD) or Long-Term Potentiation (LTP) via training Included is control of direction of the energy emission, intensity, frequency (carrier frequency and/or neuromodulation frequency), pulse duration, pulse pattern, and phase/intensity relationships to targeting and accomplishing up-regulation and/or down-regulation.

The stimulation frequency for inhibition is approximately 400 Hz or lower (depending on condition and patient). In one embodiment, the modulation frequency of lower than approximately 400 Hz is divided into pulses 0.1 to 20 msec. repeated at frequencies of 2 Hz or lower for down regulation. The stimulation frequency for excitation is in the range of approximately 600 Hz to 6 MHz. In one embodiment, the modulation frequency of higher than approximately 600 Hz. is divided into pulses 0.1 to 20 msec. repeated at frequencies higher than 2 Hz for up regulation. In this invention, the ultrasound acoustic frequency is in range of 0.3 MHz to 0.8 MHz with power generally applied less than 60 mW/cm² but also at higher target- or patient-specific levels at which no tissue damage is caused. The acoustic frequency is gated at the lower rate to impact the neuronal structures as desired (e.g., say 300 Hz for inhibition (down-regulation) or 1 kHz for excitation (up-regulation). Ultrasound therapy can be combined with therapy using other devices (e.g., Transcranial Magnetic Stimulation (TMS)).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

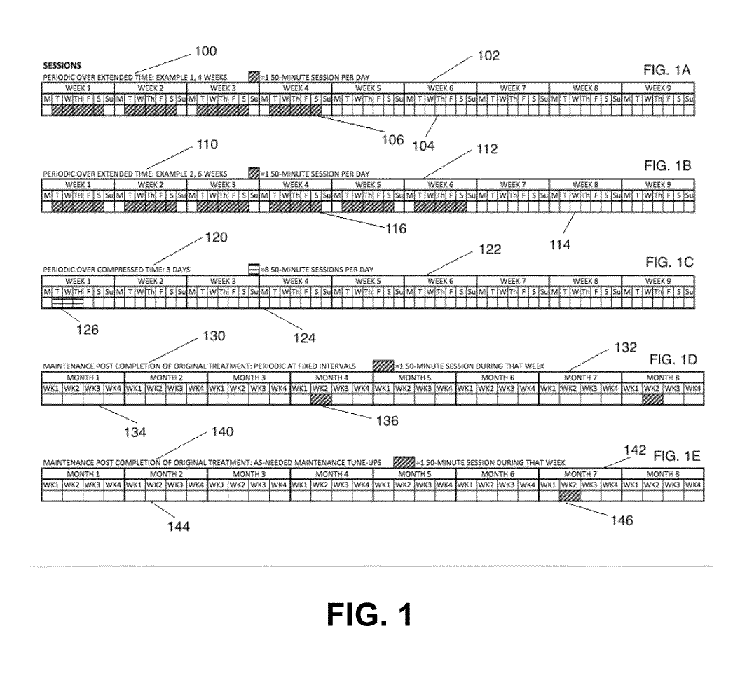

FIG. 1 shows a diagram of exemplar session types for both initial treatment and maintenance sessions. FIG. 1A illustrates example 100, Periodic Over Extended Time with 4 weeks of treatment where time divisions are weeks 102 divided into days 104 with 50-minute sessions on indicated days 106. For all of these examples, the session length could be longer or shorter than 50 minutes. FIG. 1B illustrates example 110, Periodic Over Extended Time with 6 weeks of treatment where time divisions are weeks 112 divided into days 114 with 50-minute sessions on indicated days 116. FIG. 1C illustrates example 120, Periodic Over Compressed Time with 3 days of treatment where time divisions are weeks 122 divided into days 124 with 50-minute sessions on indicated days 166. FIG. 1D illustrates example 130, Maintenance Post Completion of Original Treatment at Fixed Intervals where time divisions are months 132 divided into weeks 134 with 50-minute sessions during indicated weeks 136. FIG. 1E illustrates example 140, Maintenance Post Completion of Original Treatment with As-Needed Maintenance Tune-Ups where time divisions are months 142 divided into weeks 144 with 50-minute sessions during indicated week 146.

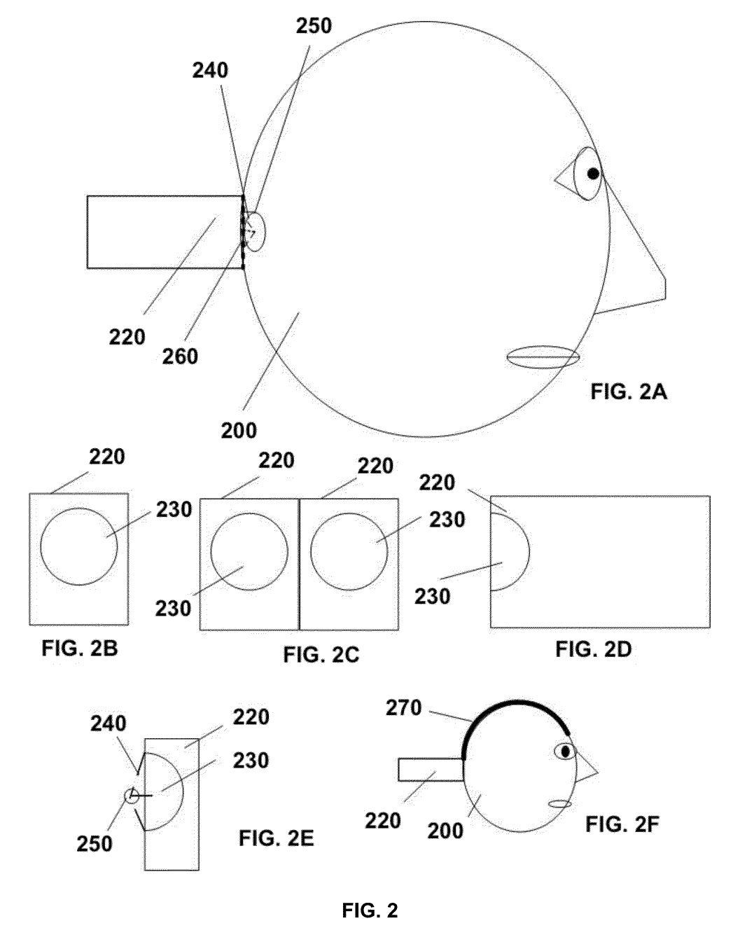

FIG. 2 shows a saggital view of the configuration for neuromodulation of the occipital nerve. Patient head 200 contains occipital nerve bundle 250. Ultrasound transducer 220 focuses sound field 240 on occipital nerve bundle 250. For the ultrasound to be effectively transmitted through intervening tissue to the neural targets, coupling must be put into place. Ultrasound transmission medium (e.g., Dermasol from California Medical Innovations or silicone oil in a containment pouch) is used as insert within the ultrasonic transducer (230 in FIGS. 2B-2E). Ultrasound gel layer 260 that provides the interface for ultrasound conduction between ultrasound transducer 220 and head 200 completes the conduction pathway.

If patient sees impact, he or she can move transducer in the X-Y direction (Z direction is along the length of transducer holder and could be adjusted as well). The elongated shape is convenient for the patient to hold and also for use with a positioning headband as shown in FIG. 2F showing patient head 100 with ultrasound transducer 220 and anterior-posterior headband 270. A hat style or open frame with side-to-side stabilization (neither shown) can be employed as alternative embodiments. Ultrasound transducer 220 is moved in and out of a holder (not shown) to provide the appropriate distance between ultrasonic transducer 220 and occipital nerve bundle target 250. In other embodiments, alternative fixed configurations, either of different ultrasonic transducer focal lengths or of different fixed positions in holders, are available for selection for specific patients.

As to X-Y position on the head, the treatment for a specific patient can be planned using physical landmarks on the patient. Loukas et al. (Loukas, M., El-Sedfy, A., Tubbs, R. S., Louis Jr., R. G., Wartmann, Ch. T., Curry, B., and R. Jordan, “Identification of greater occipital nerve landmarks for the treatment of occipital neuralgia,” Folia Morphol., Vol. 65, No. 4, pp. 337-342, 2006) used an approach that takes patient skull size into account. While the location of the Greater Occipital Nerve for anesthesia or any other neurosurgical procedure is typically viewed as “one thumb's breadth lateral to the external occipital protuberance (2 cm laterally) and approximately at the base of the thumb nail (2 cm inferior),” the study found the appropriate point was located “approximately 41% of the distance along the inter-mastoid line (medial to a mastoid process) and 22% of the distance between the external occipital protuberance and the mastoid process.” In addition, the patient can adjust positioning based on effect.

Ultrasound transducer 220 with ultrasound-conduction-medium insert 230 are shown in front view in FIG. 2B for a single transducer 220 for unilateral and in FIG. 2C for pair of transducers 220 for bilateral stimulation. A side view of the same elements in shown in FIG. 2D. FIG. 2E again shows a side view of ultrasound transducer 220 and ultrasound-conduction-medium insert 230 with ultrasound field 240 focused on the occipital nerve bundle target 250. The focus of ultrasound transducer 220 can be purely through the physical configuration of its transducer array (e.g., the radius of the array) or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

Transducer arrays of the type 220 may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. The design of the individual array elements and power applied will determine whether the ultrasound is high intensity or low intensity (or medium intensity) and because the ultrasound transducers are custom, any mechanical or electrical changes can be made, if and as required. Vendors such as Blatek and Keramos-Etalon in the U.S. and Imasonic in France can supply suitable ultrasound transducers.

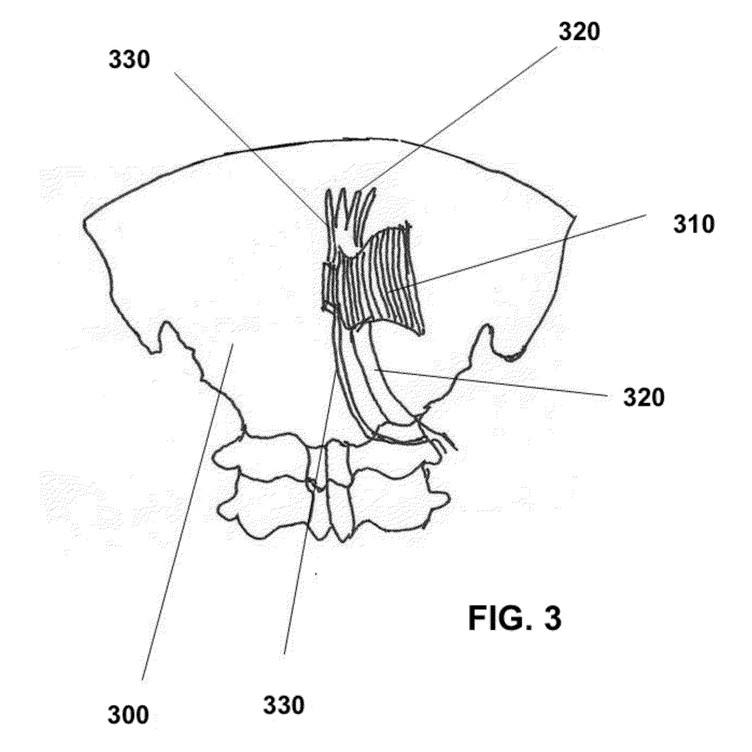

FIG. 3 anatomy of the occiput illustrating the location of occipital nerves. Occipital bone section 300 has trapezius muscle complex 310 through which the Greater Occipital Nerve 320 and the Third Occipital Nerve 330 pass. The occipital nerves occur bilaterally. Neuromodulation of which side will be most effective is headache specific and patient specific. In an alternative embodiment, bilateral neuromodulation will be supplied and this will be the usual situation. In another embodiment, the current invention will be applied to one side of the patient and an alternative treatment to the other side. Alternative invasive treatments have been electrical stimulation, local anesthetic blocks, surgical transection, surgical resection, radiofrequency, alcohol/phenol infiltration, radiosurgery, and cryotherapy. Medications and other non-invasive treatments such as avoidance of triggers, diet modification, physical therapy, chiropractic manipulation, and acupuncture have been used as well.

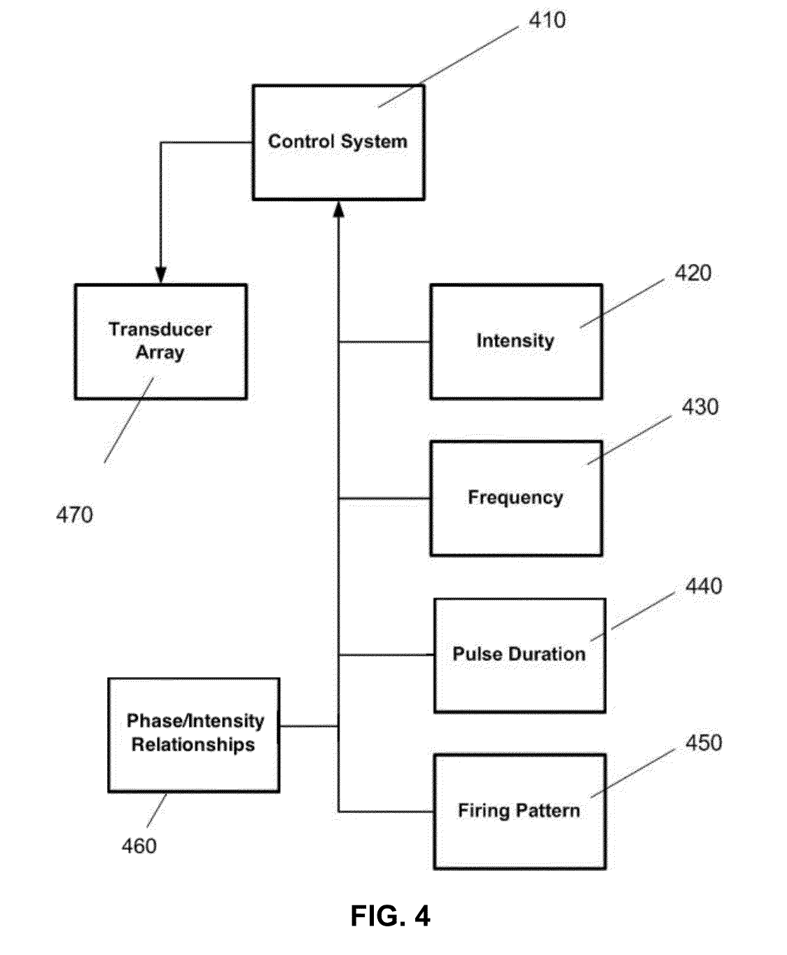

FIG. 4 illustrates the control circuit for any of these applications. Control System 410 receives its input from Intensity setting 420, Frequency setting 430, Pulse-Duration setting 440, Firing-Pattern setting 450, and Phase/Intensity Relationships 460. Control System 410 then provides output to drive Transducer Array 470 and thus deliver the neuromodulation. Settings may be input by the healthcare professional or, under the prescription and directions of a physician, set by the patient.

As indicated by previous work noted above for electrical stimulation, the positive effect of treatment, so that in addition to any acute positive effect, there will be a long-term “training effect” with Long-Term Depression (LTP) and Long-Term Potentiation (LTD) depending on the central intracranial targets to which the occipital nerve is connected.

The invention can be applied to a number of conditions including headaches in various forms, migraine headaches in various forms, cluster headaches in various forms, neuralgias, facial, and other pain or tension syndromes.

Kovacs et al. (Kovacs, S. Peeters, R., De Ridder, D., Plazier, M., Menovsky, T. and S. Sunaert, “Central Effects of Occipital Nerve Electrical Stimulation Studied by Functional Resonance Imaging,” Neuromodulation: Technology at the Neural Interface, Vol 14, Issue 1, pages 46-57, January/February 2011, Article first published online: 7 Dec. 2010 DOI: 10.1111/j.1525-1403.2010.00312.x) applied electrical stimulation of the occipital nerve and looked at the impact on neural structures as determined through fMRI. As shown in the fMRI, major areas of activation were the hypothalami, the thalami, the orbito-frontal cortex, the prefrontal cortex, periaqueductal gray, the inferior parietal lobe, and the cerebellum. As to deactivation, the major areas were in the primary motor area (M1) the primary visual area (V1), the primary auditory area (A1), and the somatosensory (S1), the amygdala, the paracentral lobule, the hippocampus, the secondary somatosensory area (S2), and the supplementary motor area (SMA). Ultrasound neuromodulation provided by the current invention would have activate and deactivate the same structures and thus can provide therapeutic effects related to the neuromodulation of those structures.

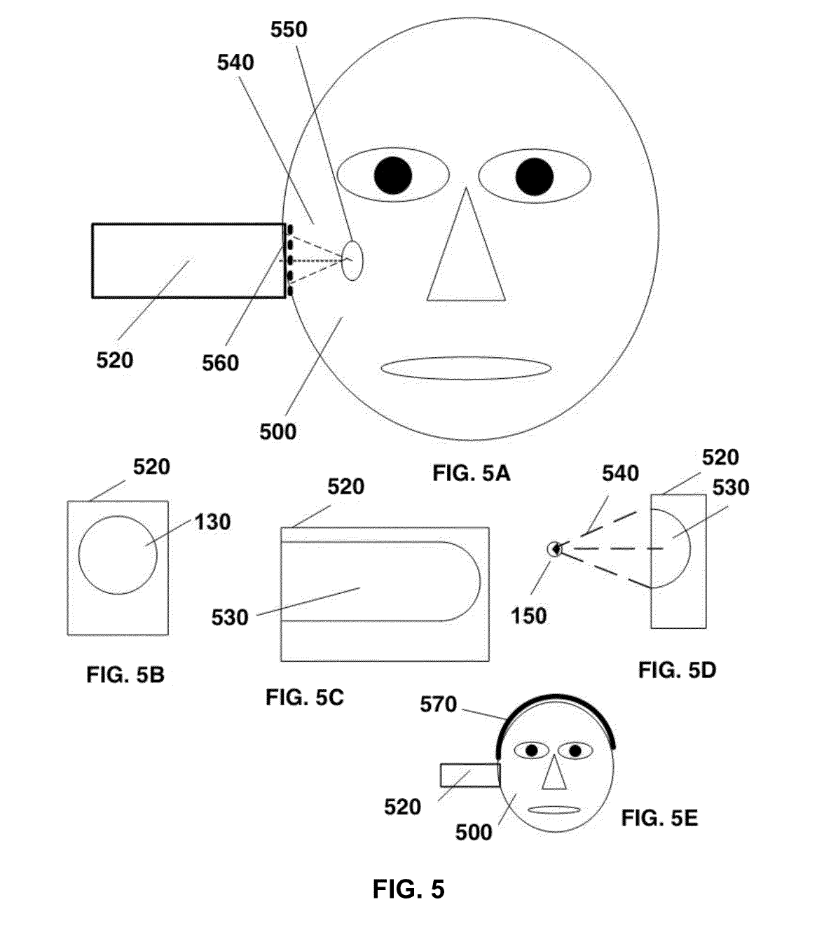

FIG. 5 shows a frontal view of the configuration for neuromodulation of the Sphenopalatine Ganglion (SPG) and related structures such as the sphenopalatine nerve and the vidian nerve. For the purpose of this discussion that the additional structures could be included. Patient head 500 contains Sphenopalatine Ganglion 550. Ultrasound transducer 520 focuses sound field 540 on Sphenopalatine Ganglion 550. For the ultrasound to be effectively transmitted through intervening tissue to the neural targets, coupling must be put into place. Ultrasound transmission medium (e.g., Dermasol from California Medical Innovations or silicone oil in a containment pouch) is used as insert within the ultrasonic transducer (530 in FIGS. 5B-5D). Ultrasound gel layer 560 that provides the interface for ultrasound conduction between ultrasound transducer 520 and head 500 completes the conduction pathway. In the illustrated embodiment, ultrasound transducer 520 is elongated to allow a longer focal length to be employed. The elongated shape is convenient for the patient to hold and also for use with a positioning headband as shown in FIG. 5E showing patient head 500 with ultrasound transducer 520 and headband 570. Ultrasound transducer 520 is moved in and out of a holder (not shown) to provide the appropriate distance between ultrasonic transducer 520 and Sphenopalatine Ganglion target 550. In other embodiments, alternative fixed configurations, either of ultrasonic transducer focal lengths or of different fixed positions in holde, are available for selection for specific patients. As to X-Y position on the head, the treatment for a specific patient can be planned using physical landmarks on the patient (for example, positioning the ultrasound transducer at lower edge of the zygomatic arch at the point anteriorly-posteriorly where the frontal process of the zygomatic bone meets the temporal process of the zygomatic bone). Alternatively, a standard x-ray examination based on bone can be done; taking an MRI or other scan is not necessary. In addition, the patient can adjust positioning based on effect. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well.

Ultrasound transducer 520 with ultrasound-conduction-medium insert 530 is shown in front view in FIG. 5B and in a side view in FIG. 5C. FIG. 5D again shows a side view of ultrasound transducer 520 and ultrasound-conduction-medium insert 530 with ultrasound field 540 focused on the Sphenopalatine Ganglion target 550. The focus of ultrasound transducer 520 can be purely through the physical configuration of its transducer array (e.g., the radius of the array) or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

Transducer arrays of the type 520 may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Blatek and Keramos-Etalon in the U.S. are other custom-transducer suppliers. The design of the individual array elements and power applied will determine whether the ultrasound is high intensity or low intensity (or medium intensity) and because the ultrasound transducers are custom, any mechanical or electrical changes can be made, if and as required.

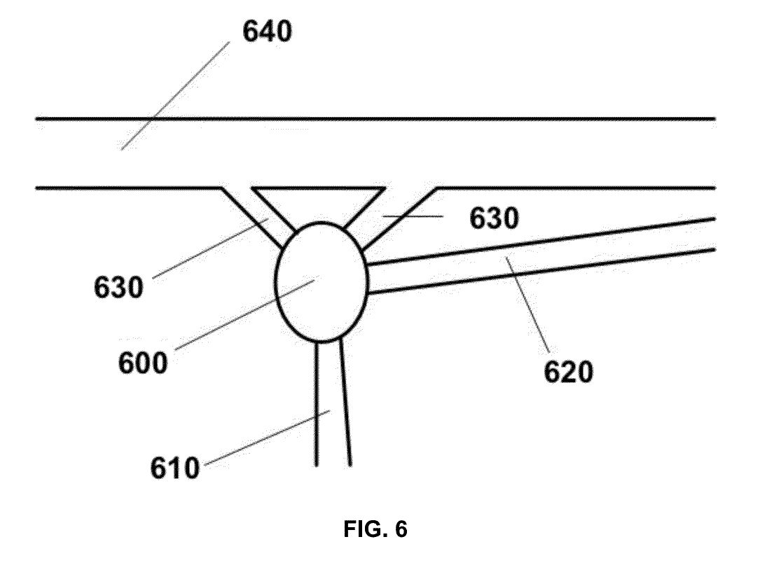

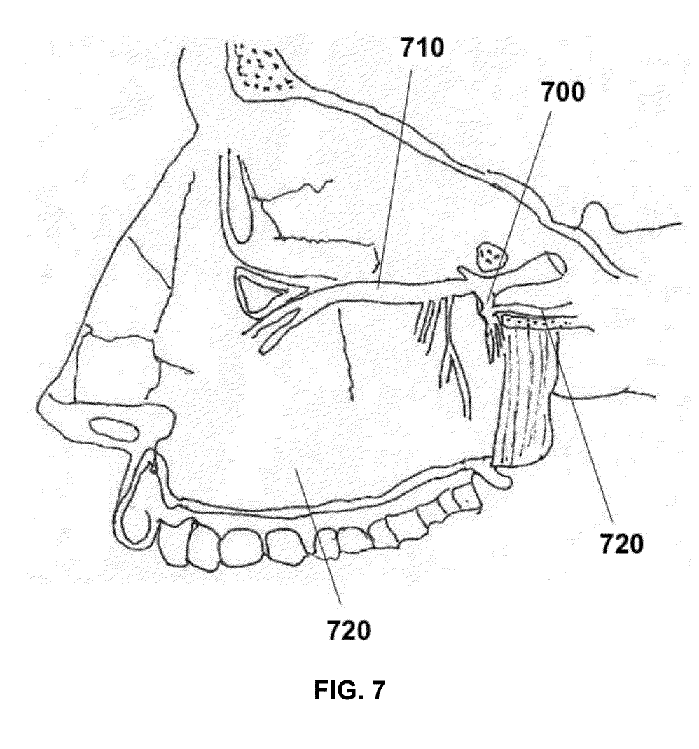

FIG. 6 shows the configuration surrounding Sphenopalatine Ganglion 600. Sphenopalatine Ganglion 600 is contained within the Sphenopalatine (or Pterygopalatine) fossa (not shown) and hangs down from maxillary nerve 640 connected to it by Sphenopalatine Nerves 630 with connections to vidian nerve 620 and palatine nerves 610. The vidian nerve 620 connects to the Sphenopalatine Ganglion 600. Vidian nerve 620 contains parasympathetic fibers (which synapse to Sphenopalatine Ganglion 600). The vidian nerve also contains sympathetic fibers and sensory fibers, transmitting sensation from part of the nasal septum. The sphenopalatine nerves 630 are sensory nerves physically connect the Sphenopalatine Ganglion 600 to the maxillary nerve 640, but pass through and do not synapse with Sphenopalatine Ganglion 600. These structures are located bilaterally. Neuromodulation of which side will be most effective is headache specific and patient specific. In an alternative embodiment, bilateral neuromodulation will be supplied. In another embodiment, the current invention will be applied to one side of the patient and an alternative treatment to the other side. Alternative invasive treatments have been electrical stimulation, local anesthetic blocks, surgical transection, surgical resection, radiofrequency, alcohol/phenol infiltration, radiosurgery, and cryotherapy. Medications and other non-invasive treatments such as avoidance of triggers, diet modification, physical therapy, chiropractic manipulation, and acupuncture have been used as well. FIG. 7 shows selected physical relationships with anterior skull 700 showing Sphenopalatine Ganglion 710, maxillary nerve 720, and vidian nerve 730. As with all the clinical treatments, the control systems is shown in FIG. 4.

While the parasympathetic nervous system is subject to Long-Term Potentiation (LTP) such that in addition to the acute effect that there is the potential for a long-term training effect, there can be Long-Term Potentiation (LTP) and Long-Term Depression (LTD) at the intracranial targets to which the Sphenopalatine Ganglion and associated neural structures are attached.

The invention can be applied to a number of conditions including headaches in various forms, migraine headaches in various forms, cluster headaches in various forms, neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders. It can also be applied to modification of the properties of the blood-brain Barrier and cerebral blood flow.

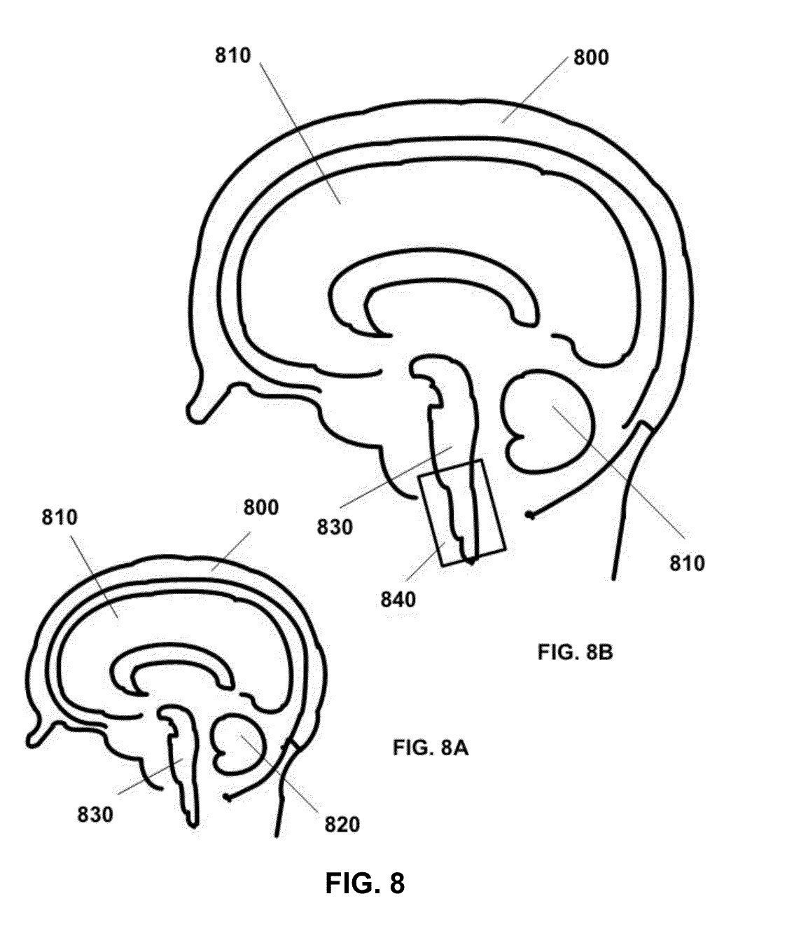

FIG. 8 shows sagittal view of brain highlighting the Reticular Activating System (RAS) 830 including skull 800 with cerebrum 810 along with cerebellum 820. FIG. 8B again shows the Reticular Activating System 830 including skull 800 with cerebrum 810 along with cerebellum 820, but this time with ultrasound transducer 840 approximately aligned along the axis of the Reticular Activating System and placed against the neck. The ultrasound transducer 840 does not cover the entire length of the Reticular Activating System (RAS) first because the upper part of the is not physically accessible (although the top of the outline 830 is the midbrain which is outside the RAS) and second because the ultrasound field can be steered to a point above the top of the ultrasound transducer 840. In another embodiment, the ultrasound transducer is perturbed laterally, up and down, and/or in and out causing enhanced change in the target neural tissue.

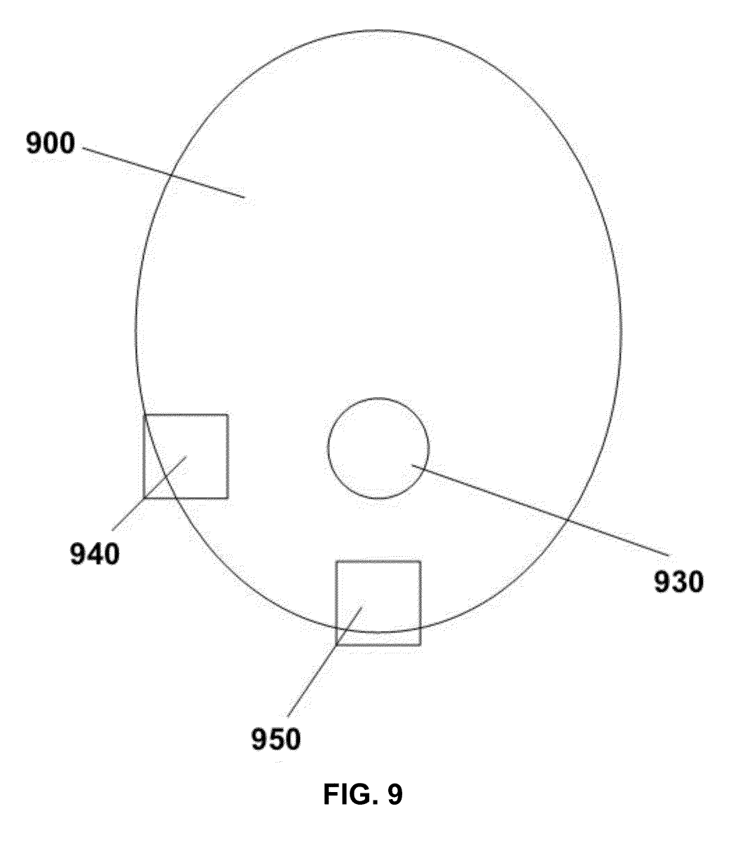

FIG. 9 shows the top view of patient head 900 showing two embodiments of ultrasound transducer placements with respect to Reticular Activating System 930, the first in which the ultrasound transducer is placed laterally 940 to RAS 930 and against the patient's neck and the second in which the ultrasound transducer 950 is placed posterior to RAS 930 against the patient's neck. Note that the placement of lateral ultrasound transducer 930 can be to the right of RAS 930 or to its left. For the ultrasound to be effectively transmitted through the tissues to the RAS target, coupling must be put into place. Ultrasound transmission medium (e.g., Dermasol from California Medical Innovations or silicone oil in a containment pouch) (not shown) is interposed with one mechanical interface to the ultrasound transducer, either 940 or 950 completed by a layer of ultrasound transmission gel (not shown). The depth of the point where the ultrasound is focused depends on the shape of the transducer and setting of the phase and amplitude relationships of the elements of the ultrasound transducer array discussed in relation to the Control Circuit in FIG. 4. In other embodiments, ultrasound transducers may be place on both sides of the patient's neck. In a further embodiment, multiple ultrasound transducers may be used either in the vertical direction, horizontal direction, or both.

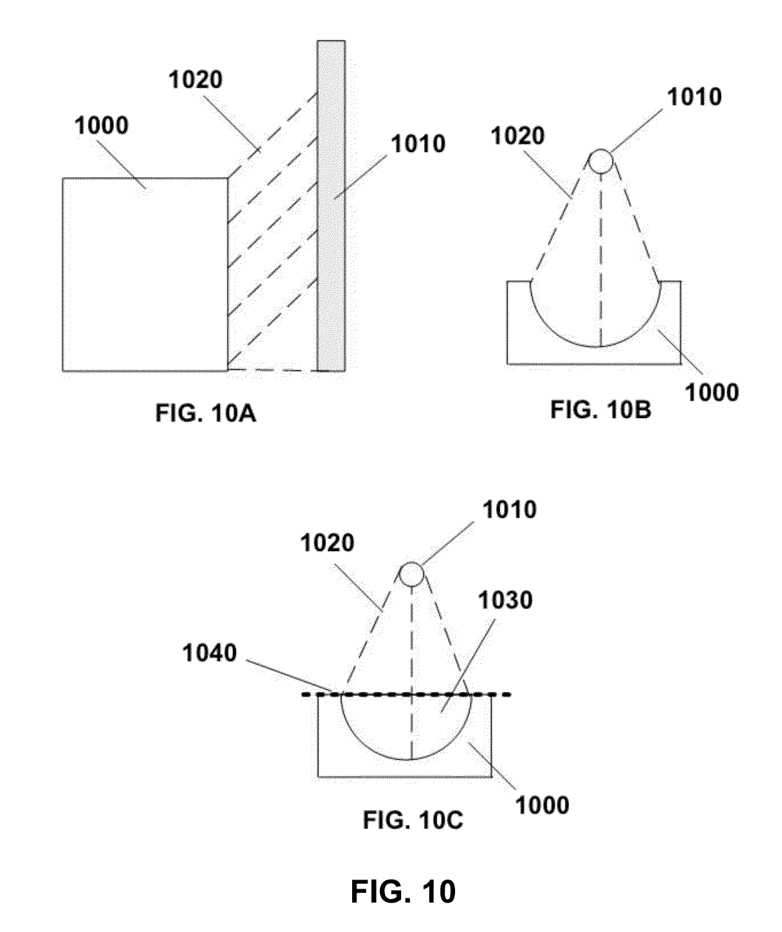

FIG. 10A shows a lateral view of ultrasound transducer 1000 with its ultrasound pattern 1020 aimed at the Reticular Activating System (RAS) target 1010. Ultrasound-transducer field 1020 is steered upwards by controlling the phase/intensity relationships of the array elements of ultrasound transducer 1000 so it can hit target 1010 at a point that is superior to the top of ultrasound transducer 1000. Transducer array assemblies of this type may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Blatek and Keramos-Etalon in the U.S. are other custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations. FIG. 10B shows a plan view of the configuration with ultrasound transducer 1000, RAS target 1010, and ultrasound pattern 1020. FIG. 10C shows the configuration where added to ultrasound transducer 1000, RAS target 1010, and ultrasound pattern 1020 are ultrasound conduction medium 1030 contained within the transducer, and ultrasound conducting gel layer 1040 which is pressed against the skin of the patient. In another embodiment, a plurality of ultrasonic transducers is aimed at the Reticular Activating System.

In still another embodiment movement of the transducer and/or controlling stimulation parameters and seeing the physiological response of the patient is used to correctly locate the Reticular Activating System.

The invention can be applied for a variety of clinical purposes such as reversibly putting a patient to sleep or waking them up (for example, for the purpose of anesthesia) or reversibly putting a patient into a coma (for example for the purpose of protecting or rehabilitating the brain of the patient after a stroke or head injury). Effects can be either acute or durable effect through Long-Term Potentiation (LTP) and/or Long-Term Depression (LTD). Since the effect is reversible putting the patient in even a vegetative state is safe if handled correctly. The application of LTP or LTD provides a mechanism for adjusting the bias of patient activity up or down. Appropriate radial (in-out) positions can be determined through patient-specific imaging (e.g., PET or fMRI) or set based on measurements to the mid-line. The positions can set manually or via a motor (not shown). The invention allows stimulation adjustments in variables such as, but not limited to, intensity, firing pattern, pulse duration, frequency, phase/intensity relationships, dynamic sweeps, and position.

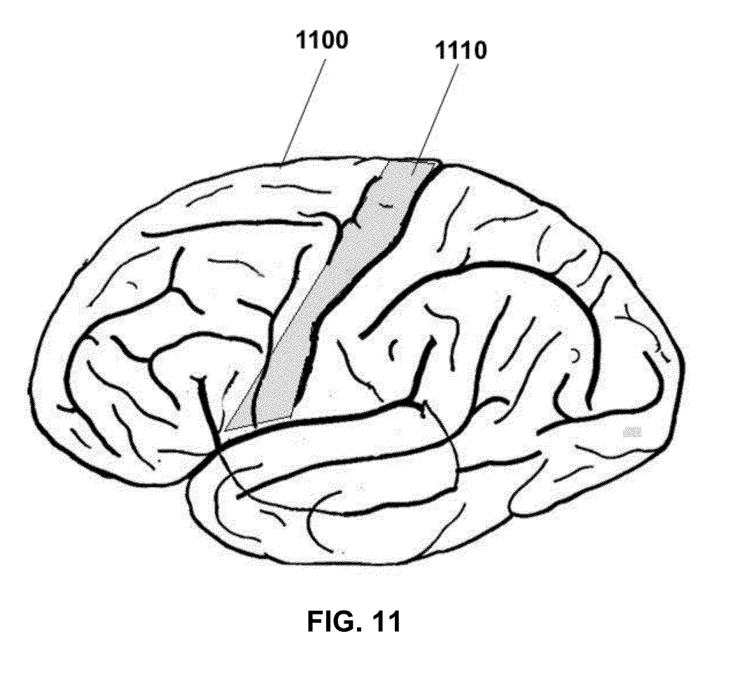

FIG. 11 shows a brain 1100 with cerebral folds with shaded area 1110 denoting the location of the Primary Motor Cortex (designated as M1). The intervening bony skull is not shown. All or part of the motor cortex can be damaged by a stroke and other areas may be damaged by ischemic or hemorrhagic stroke as well. Typically the edges of an area impacted by a stroke are viable and neuromodulation of these edges can mitigate against further loss of tissue acutely. In the longer term, neuromodulation of this viable tissue can foster post-stroke rehabilitation. Besides Primary Motor Cortex, strokes cause lesions in the Primary Sensory Cortex, Wernicke's Area, posterior limb of internal capsule, basis pontis, corona radiate, and other neural centers.

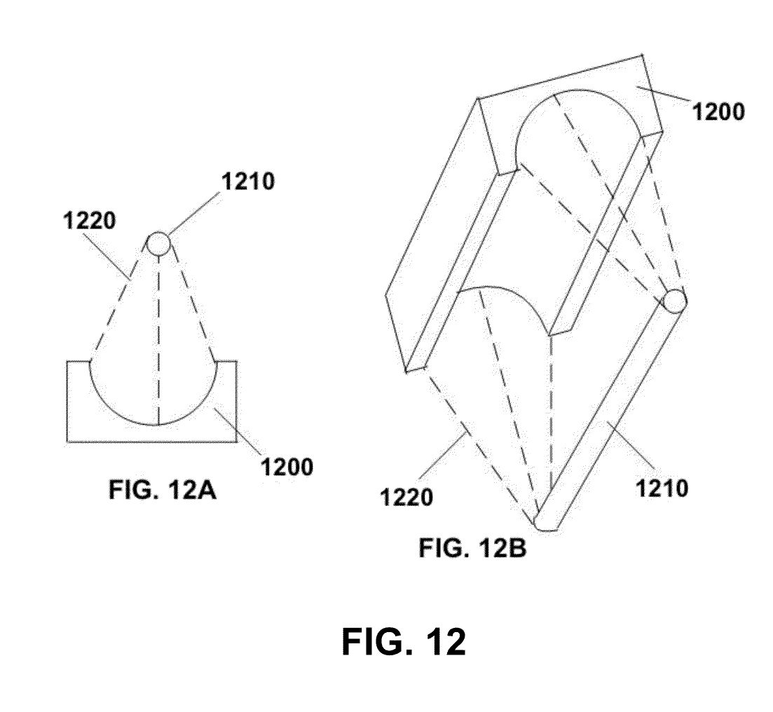

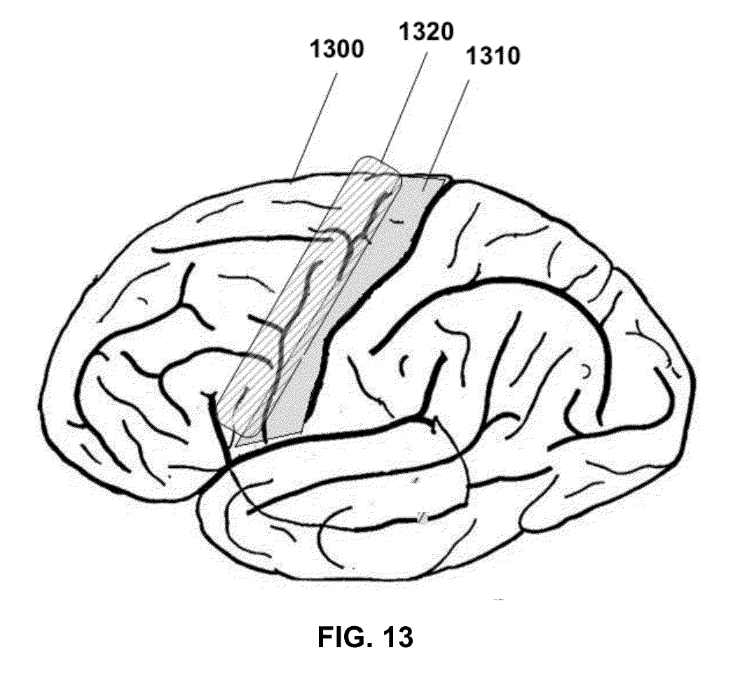

FIG. 12 shows an ultrasound transducer array configured to produce an elongated pencil-shaped focused field. Such an array would he applied to stimulate an elongated target such as the motor cortex. Note that one embodiment is a swept-beam transducer with the capability of sweeping the sound field over any portion of the length of the ultrasound transducer. Thus it is possible to determine over what length of a target that the ultrasound is applied. For example, one could apply ultrasound to only the superior portion of the target. In FIG. 12A, an end view of the array is shown with curved-cross section ultrasonic array 1200 forming a sound field 1220 focused on target 1210. FIG. 12B shows the same array in a side view, again with ultrasound array 1200, target 1210, and focused field 1220. FIG. 13 shows for brain 1300, the positioning of an ultrasound transducer 1320 over Primary Motor Cortex 1310. The intervening bony skull is not shown. The space between the surface of the ultrasound transducer and the surface of the head Is filled with ultrasound conduction medium (e.g., Dermasol from California Medical Innovations)(not shown) with a layer of ultrasound conduction gel between the surface of the ultrasound conduction medium and the surface of the head. One or more such ultrasound transducers may be aimed at other areas of the brain damaged by stroke. Stimulation can be unilateral or bilateral. It has been found using rTMS that there can be advantages to exciting the motor cortex ipsilateral to the brain lesion and inhibiting the motor cortex contralateral to the brain region.

Results with ultrasound neuromodulation would reflect what happens with Transcranial Magnetic Stimulation (TMS), but with the additional advantage of ultrasound neuromodulation being more focused. With respect to language, post-stroke aphasia has been successfully treated with 1 Hz rTMS to language sites (Barwood C H, Murdoch B E, Whelan B M, Lloyd D, Riek S, O'Sullivan J D, Coulthard A, Wong A., “Improved language performance subsequent to low-frequency rTMS in patients with chronic non-fluent aphasia post-stroke,” Eur J Neurol. 2010 Dec. 7. doi: 10.1111/j.1468-1331.2010.03284.x) resulting in improved naming performance, expressive language, and auditory comprehensive. Stimulation of Wernicke's area at 1 Hz in two patients with sensory dominant aphasia showed improvement when combined with language therapy (Kakuda W, Abo M, Uruma G, Kaito N, Watanabe M., “Low-frequency rTMS with language therapy over a 3-month period for sensory-dominant aphasia: case series of two post-stroke Japanese patients,” Brain Inj. 2010; 24(9):1113-7).

Motor function has been successfully improved with high-frequency rTMS stimulation to the motor cortex in terms of motor recovery (Chang W H, Kim Y H, Bang O Y, Kim S T, Park Y H, Lee P K., “Long-term effects of rTMS on motor recovery in patients after subacute stroke,” J Rehabil Med. 2010 September; 42(8):758-64), motor disability and dysphagia (Khedr E M, Fetoh N A., “Short- and long-term effect of rTMS on motor function recovery after ischemic stroke,” Restor Neurol Neurosci. 2010; 28(4):545-59), upper limb function in combination with DCS stimulation (Nowak D A, Bösl K, Podubeckà J, Carey J R., “Non-invasive brain stimulation and motor recovery after stroke,” Restor Neurol Neurosci. 2010; 28(4):531-44), upper limb with 5 Hz rTMS combined with extensor motor training, improved motor function and decreased spasticity with 1 Hz rTMS stimulation of the contralesional cerebral hemisphere followed by intense occupational therapy (Kakuda W, Abo M, Kobayashi K, Momosaki R, Yokoi A, Fukuda A, Ishikawa A, Ito H, Tominaga A., (Low-frequency repetitive transcranial magnetic stimulation and intensive occupational therapy for post-stroke patients with upper limb hemiparesis: preliminary study of a 15-day protocol,” Int J Rehabil Res. 2010 Jul. 6).

Ipsilateral primary motor cortical stimulation at 10 Hz in patients with problems with dexterity of the hand after stroke showed improvement of index finger and hand tapping in those with subcortical stroke; there was some deterioration in those with cortical stroke (Ameli M, Grefkes C, Kemper F, Riegg F P, Rehme A K, Karbe H, Fink G R, Nowak D A., “Differential effects of high-frequency repetitive transcranial magnetic stimulation over ipsilesional primary motor cortex in cortical and subcortical middle cerebral artery stroke,” Ann Neurol. 2009 September; 66(3):298-309).

In patients with impaired upper-limb function, stimulation of the primary motor cortex with Theta Burst Stimulation (TBS) demonstrated increased excitability with intermittent TBS on the same side, but decreased excitability on the side with continuous TBS of the contralesional M1 (Ackerley S J, Stinear C M, Barber P A, Byblow W D., “Combining theta burst stimulation with training after subcortical stroke,” Stroke. 2010 July; 41(7):1568-72. Epub 2010 May 20). Note that the lateral resulted in an overall decrease in upper-limb function.

In patients suffering from acute ischemic stroke, rTMS was done with one group treated at 1 Hz and the other 3 Hz; at 3 months, 1 Hz group demonstrated both decreased excitability of the non-stroke hemisphere and increased excitability of the stroke hemisphere while the 3 Hz group showed only increased excitability of the stroke hemisphere (Khedr E M, Abdel-Fadeil M R, Farghali A, Qaid M., “Role of 1 and 3 Hz repetitive transcranial magnetic stimulation on motor function recovery after acute ischaemic stroke,” Eur J Neurol. 2009 December; 16(12):1323-30. Epub 2009 Sep. 23).

There is support for the concept of intrahemispheric balance with 5 Hz rTMS stimulation ipsilaterally or 1 Hz. Stimulation contralaterally (Emara T H, Moustafa R R, Elnahas N M, Elganzoury A M, Abdo T A, Mohamed S A, Eletribi M A., “Repetitive transcranial magnetic stimulation at 1 Hz and 5 Hz produces sustained improvement in motor function and disability after ischaemic stroke,” Eur J Neurol. 2010 September; 17(9):1203-9. Epub 2010 Apr. 8).

rTMS stimulation at 3 Hz for the esophageal motor cortex in patients with dysphagia resulting from acute lateral medullary infarction (LMI) or other brainstem infarctions resulted in swallowing maintained over two months (Khedr E M and Abo-Elfetoh N., “Therapeutic role of rTMS on recovery of dysphagia in patients with lateral medullary syndrome and brainstem infarction,” J Neurol Neurosurg Psychiatry. 2010 May; 81(5):495-9. Epub 2009 Oct. 14.).

Stroke patients may benefit from other interventions as well. For example, rTMS stimulation of the Left Dorsolateral Prefrontal cortex (DLPFC) if the affected side had a positive effect on mode in terms of decreased depression even though there was no impact on cognition, Kim et al. (Kim B R, Kim D Y, Chun M H, Yi J H, Kwon J S., “Effect of repetitive transcranial magnetic stimulation on cognition and mood in stroke patients: a double-blind, sham-controlled trial,” Am J Phys Med Rehabil. 2010 May; 89(5):362-8).

Ultrasound transmission medium (e.g., Dermasol from California Medical Innovations or silicone oil in a containment pouch) is used as insert within the ultrasonic transducer. An layer of ultrasound conduction gel is placed between the face of the transducer or an associated lens and the surface of the head or the patient being imaged. The depth of the point where the ultrasound is focused depends on the shape of the transducer and setting of the phase and amplitude relationships of the elements of the ultrasound transducer array.

A distinct advantage of ultrasound neuromodulation is the small size of the device itself and low power requirements (for example, with respect to the apparatus required for Transcranial Magnetic Stimulation and power required to use it). Since ultrasound neuromodulation devices can easily be made portable, they can be practically used at home and convalescent facilities.

The location of the stroke is immaterial from the perspective of neuromodulation. It can be applied to strokes located in cortical, subcortical, brainstem, and other regions. The region impacted by stroke can be a single one such as a large infarct or multiple small ones. It also does not matter whether the stroke is ischemic and hemorrhagic. Not only does neuromodulation foster metabolic changes, the repetitive neuromodulation can retrain neural pathways to allow restore function.

Stimulation can be done unilaterally or bilaterally to see diagnostically which muscle or muscle groups are affected. Therapeutically, the ultrasound neuromodulation can be used to stimulate muscles to exercise them.

Another consideration is combination with neuromodulation of regions other than Motor Cortex. For example, neuromodulation of the Reticular Activating System to keep the general level of brain and base central activity up to prevent Central Nervous System failure.

Again, transducer array assemblies of the appropriate type may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon and Blatek in the U.S. are other custom-transducer suppliers. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations. The Control System is shown in FIG. 4.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

The invention can be applied for a variety of stroke-related clinical purposes such as reversibly putting a patient into a coma (for example for the purpose of protecting the brain of the patient after a stroke or head injury). Effects can be either acute or durable effect through Long-Term Potentiation (LTP) and/or Long-Term Depression (LTD). Since the effect is reversible putting the patient in even a vegetative state is safe if handled correctly. The application of LTP or LTD provides a mechanism for adjusting the bias of patient activity up or down. Appropriate radial (in-out) positions can be determined through patient-specific imaging (e.g., PET or fMRI) or set based on measurements to the mid-line. The positions can set manually or via a motor (not shown). The invention allows stimulation adjustments in variables such as, but not limited to, intensity, firing pattern, frequency, phase/intensity relationships, dynamic sweeps, and position.

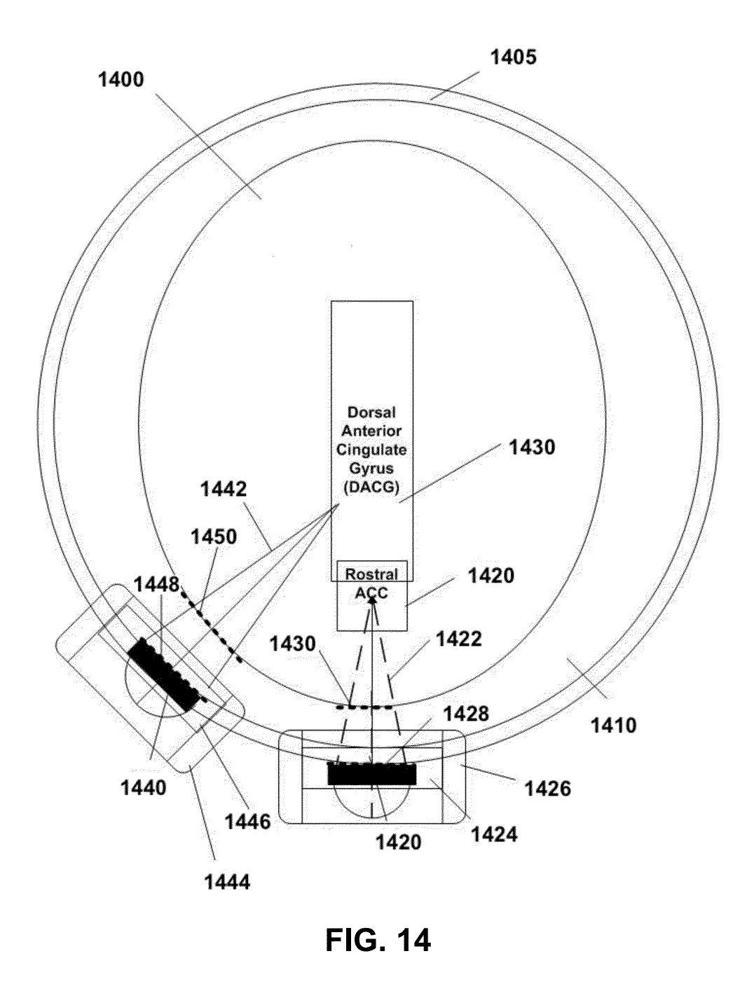

FIG. 14 shows two ultrasound transducers targeting pain-related targets. The head 1400 contains the two targets, Rostral Anterior Cingulate Cortex (ACC) 1420 and Dorsal Anterior Cingulate Gyms (DACG) 1430. These targets are known to be involved in pain processing and can be down regulated at a frequency on the order of 1 Hz. Beams from ultrasound transducers 1420 and 1440 that are fixed to track 1405 hit these targets. These are beam 1422 from ultrasound transducer 1420 and beam 1442 from ultrasound transducer 1440. Transducer 1420 mounted on support 1424 is moved radially in or out of holder 1426 by a motor (not shown) to the correct position for targeting Rostral Anterior Cingulate Cortex (RACC) 1420 under control of treatment planning software or manual control. In like manner, transducer 1440 mounted on support 1446 is moved radially in or out of holder 1444 by a motor (not shown) to the correct position for targeting Dorsal Anterior Cingulate Gyms (DACG) 1430 under control of treatment planning software or manual control. For the ultrasound to be effectively transmitted to and through the skull and to brain targets, coupling must be put into place. Ultrasound transmission medium 1410 is interposed with one mechanical interface to the ultrasound transducers 1420 and 1440 (completed by a layers of ultrasound transmission gels 1428 and 1448 on the transducer side and 1430 and 1450 on the head side). In other embodiments other neural targets known to be involved in pain processing such as the orbitofrontal cortex, insula, amygdalae, thalamus, hypothalamus, and hippocampus can be neuromodulated combined with or substituted for the Rostral Anterior Cingulate Cortex (RACC) or the Dorsal Anterior Cingulate Gyms (DACG). Depending on the given target different frequencies up to 20 Hz. may be applicable.

Transducer array assemblies of this type may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon in the U.S. is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations. FIG. 4 shows the control circuit.

The invention can be applied for a variety of clinical purposes such as treatment of acute or chronic post-operative pain, acute or chronic pain related to dental procedures, chronic pain related to conditions like fibromyalgia, low-back pain, headache, neurogenic pain, cancer pain, arthritis pain, and psychogenic pain. Effects can be either acute or durable effect through Long-Term Potentiation (LTP) and/or Long-Term Depression (LTD). Appropriate radial (in-out) positions can be determined through patient-specific imaging (e.g., PET or fMRI) or set based on measurements to the mid-line. The positions can set manually or via a motor (not shown). The invention allows stimulation adjustments in variables such as, but not limited to, intensity, firing pattern, frequency, pulse duration, phase/intensity relationships, dynamic sweeps, and position.

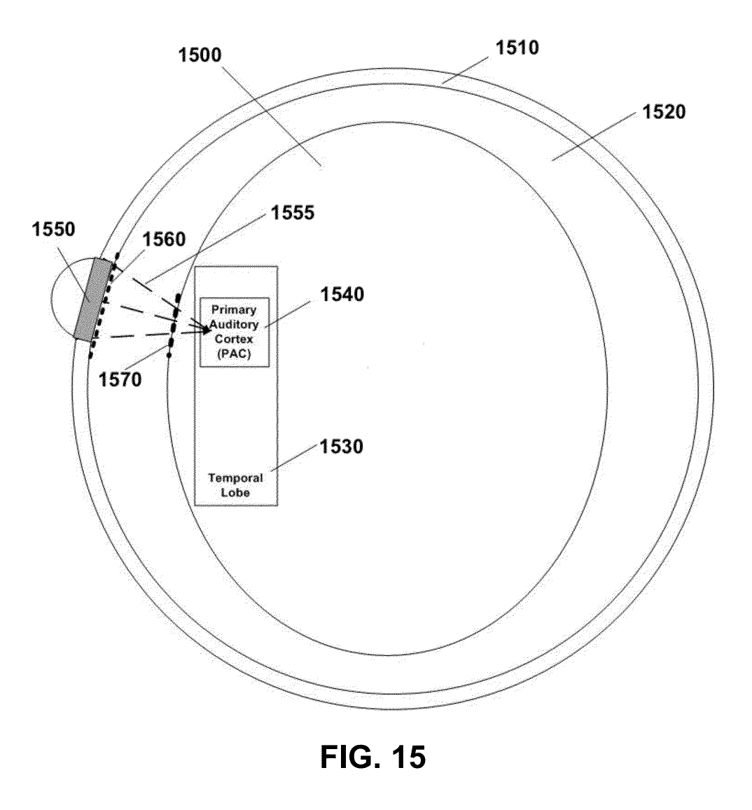

FIG. 15 shows an ultrasound transducer targeting to treat tinnitus. The head 1500 contains the tinnitus target, the Primary Auditory Cortex 1540 that is located within the Temporal Lobe 1530. The beam 1555 from ultrasound transducer 1550 that is fixed to track 1510 to hit this target 1540. For the ultrasound to be effectively transmitted to and through the skull and to brain targets, coupling must be put into place. Ultrasound transmission medium (for example Dermasol from California Medical Innovations) 1520 is interposed with one mechanical interface to the ultrasound transducer 1550 and the other mechanical interface to head 1500 (completed by a layers of ultrasound transmission gel 1560 on the transducer side and 1570 on the head side). Again FIG. 4 shows the control circuit.

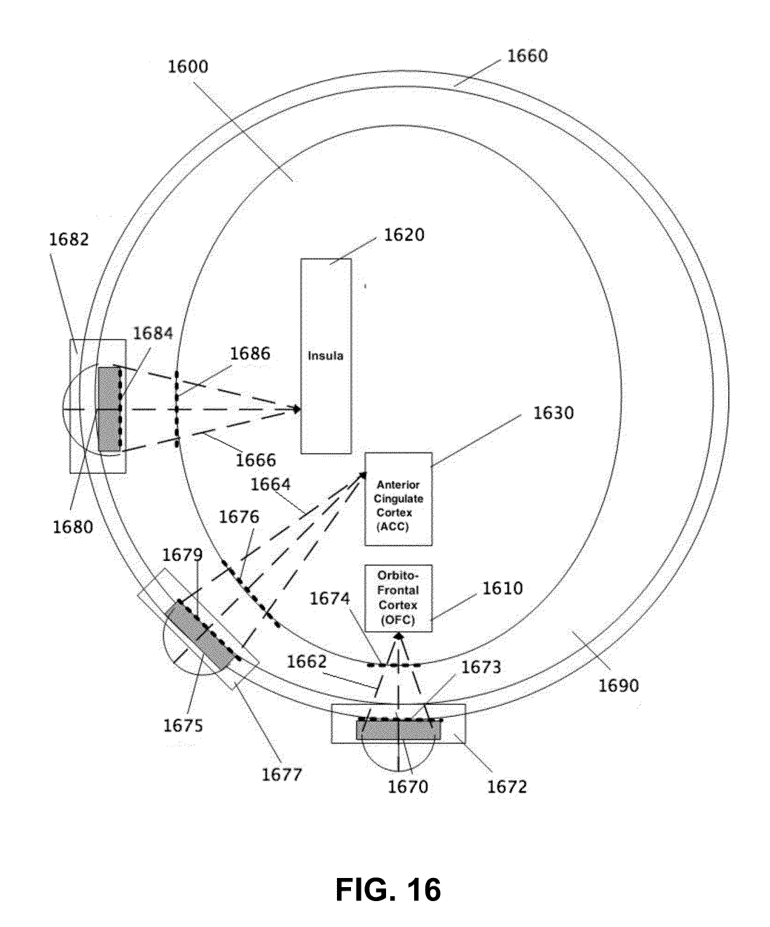

FIG. 16 shows a set of ultrasound transducers targeting to treat PTSD. The head 1600 contains the five targets, Orbito-Frontal Cortex (OFC) 1620, Anterior Cingulate Cortex (ACC) 1630, Insula 1640, Amygdala 1650, and Hippocampus 1660. Note that while these five targets are covered here, fewer can work as well, or an addition or substitution of other targets such as the Ventro-Medial Pre-Frontal Cortex can work also. These targets are hit by ultrasound from transducers 1622, 1632, 1642, 1652, and 1662, fixed to track 1605. Ultrasound transducer 1622 with its beam 1624 is shown targeting the OFC 1620 which would be up regulated, transducer 1632 with its beam 1634 is shown targeting the ACC 1630 which would be up regulated, transducer 1642 with its beam 1644 is shown targeting the Insula 1640 which would be down regulated, transducer 1652 with its beam 1654 is shown targeting the Amygdala 1640 which would be down regulated, and transducer 1662 with its beam 1664 is shown targeting the Hippocampus 1660 which would be up regulated. For ultrasound to be effectively transmitted to and through the skull and to brain targets, coupling must be put into place. Ultrasound transmission (for example Dermasol from California Medical Innovations) medium 1608 is interposed with one mechanical interface to the frame 1605 and ultrasound transducers 1622, 1632, 1642, 1652, and 1662 (completed by a layer of ultrasound transmission gel 1610) and the other mechanical interface to the head 1600 (completed by a layer of ultrasound transmission gel 1612). In another embodiment the ultrasound transmission gel is only placed at the particular places where the ultrasonic beams from the transducers are located rather than around the entire frame and entire head. FIG. 4 again shows the control circuit.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What I claim is:

1. A method of non-invasively neuromodulating the target occipital nerves using ultrasound stimulation, the method comprising:
aiming an ultrasound transducer at the target,
applying pulsed power to said ultrasound transducer via a control circuit thereby modulating the activity of the target, and
applying the neuromodulation in sessions,
whereby therapeutic results are obtained.

2. The method of claim 1, wherein the neural target is comprised of one or a plurality of the occipital nerves.

3. The method of claim 2, wherein the disorder treated is selected from the group consisting of headaches in various forms, migraine headaches in various forms, cluster headaches in various forms, neuralgias, facial and other pain or tension syndromes.

4. The method of claim 2, wherein the ultrasound neuromodulation results in activation of the hypothalami, the thalami, the orbito-frontal cortex, the prefrontal cortex, periaqueductal gray, the inferior parietal lobe, and the cerebellum.

5. The method of claim 2, wherein the ultrasound neuromodulation results in deactivation of the primary motor area (M1) the primary visual area (V1), the primary auditory area (A1), and the somatosensory (S1), the amygdala, the paracentral lobule, the hippocampus, the secondary somatosensory area (S2), and the supplementary motor area (SMA).

6. The method of claim 2, wherein the neuromodulation of the occipital nerves is selected from the group consisting of unilateral and bilateral.

7. The method of claim 1, further comprising focusing the sound field of an ultrasound transducer at the target Sphenopalatine Ganglion and associated structures neuromodulating the activity of the target in a manner selected from the group of up-regulation, down-regulation.

8. The method of claim 7, wherein the disorder treated is selected from the group consisting of headaches in various forms, migraine headaches in various forms, cluster headaches in various forms, neuralgias, other pain syndromes, movement and muscular disorders, epilepsy, hypertension, cerebral vascular disorders including stroke, autoimmune diseases, sleep disorders, asthma, metabolic disorders, addiction, autonomic disorders (including, but not limited to cardiovascular disorders, gastrointestinal disorders, genitourinary disorders), and neuropsychiatric disorders.

9. The method of claim 1 where the neural structure is the Reticular Activating System, wherein the neuromodulation manner is selected from the group of up-regulation and down-regulation.

10. The method of claim 9, wherein the clinical function is selected from the group consisting of: reversibly putting a patient to sleep or waking them up and reversibly putting a patient into a coma.

11. The method of claim 9, wherein the clinical purpose is selected from the group consisting of: anesthesia, protecting or rehabilitating the brain after a stroke, and protecting or rehabilitating the brain after head trauma.

12. The method of claim 1 wherein the condition treated is selected from the group consisting of stroke mitigation and stroke rehabilitation.

13. The method of claim 12, wherein the one or a plurality of stroke-related targets is selected from the group consisting Primary Motor Cortex, Primary Sensory Cortex, Wernicke's Area, posterior limb of internal capsule, basis pontis, corona radiate, and other neural centers.

14. The method of claim 12, wherein the clinical function is selected from the group consisting of exciting the motor cortex ipsilateral to the brain lesion and inhibiting the motor cortex contralateral to the brain region.

15. The method of claim 12, wherein the location of the stroke is selected from the group consisting of cortical, subcortical, and brainstem.

16. The method of claim 12, wherein the cause of the stroke is selected from the group consisting of ischemic and hemorrhagic.

17. The method of claim 12 where neuromodulation for stroke is combined with the neuromodulation of the Reticular Activating System to keep the general level of brain and base central activity up to prevent Central Nervous System failure.

18. The method of claim 1, wherein the step of aiming comprising orienting the ultrasound transducer and focusing the ultrasound so that it hits one or a plurality of pain-related neural targets selected from the group consisting of orbitofrontal cortex, Rostral Anterior Cingulate Cortex and Dorsal Anterior Cingulate Gyms, insula, amygdala, thalamus, hypothalamus, and hippocampus.

19. The method of claim 1, further comprising aiming an ultrasound transducer neuromodulating tinnitus-related neural targets in a manner selected from the group of up-regulation, down-regulation.

20. The method of claim 1, wherein one or a plurality of Post Traumatic Syndrome Disorders-related targets are selected from the group consisting of Orbito-Frontal Cortex, Anterior Cingulate Cortex, Insula, Amygdala, and Hippocampus.
